(19) United States
(12) Patent Application Publication
(10) Pub. No.: US 2002/0133115 A1
(43) Pub. Date: Sep. 19, 2002

(54) Apparatus and methods for capture of medical agents

(75) Inventors: Lucas S. Gordon, Redmond, WA (US); Mary Jo Gordon, Carnation, WA (US); Lawrence Kamm, San Diego, CA (US)

Correspondence Address: LYON & LYON LLP, 633 WEST FIFTH STREET, SUITE 4700, LOS ANGELES, CA, 90071, US

(73) Assignee: Pharmaspec Corporation

(21) Appl. No.: 09/973,620

(22) Filed: Oct. 9, 2001

Related U.S. Application Data
(60) Provisional application No. 60/275,773, filed on Mar. 13, 2001.

(51) Int. Cl. A61M 29/00

(52) U.S. Cl. 604/96.01; 604/523; 604/246; 604/522; 607/103

(57) ABSTRACT

The present invention relates to catheter devices and methods for their use to modulate medical agents at selected locations in a patient's body.

### RELATED APPLICATIONS

This application is related to and claims priority from U.S. Provisional Patent Application Serial No. 60/275,773, which is incorporated, including any drawings, as if fully set forth herein.

### FIELD OF THE INVENTION

The present invention pertains to the fields of medicine and medical devices. In particular it relates to catheter devices and methods for their use to modulate the quantity, location and activity of medical agents in a patient's body.

### BACKGROUND OF THE INVENTION

The following is offered as background information only and is not considered, nor is it to be construed as, prior art to the present invention.

Physicians today have at their disposal a vast arsenal of extremely powerful therapeutic and diagnostic medical agents. However, the very power that imbues these agents with great curative and diagnostic utility carries with it a potentially serious downside. That is, while being efficacious in achieving their intended results at a particular location or organ in a patient's body, these medical agents may be toxic in non-targeted areas or organs. To partially assuage this undesirable side effect, numerous means for localized delivery of medical agents have been reported.

For instance, Corday et al., in U.S. Pat. No. 4,689,041, describe a method for delivering medical agents to a region of a patient's heart made inaccessible by an occluded artery by retrograde (opposite the direction of normal blood flow) venous perfusion. Corday employs a catheter having a balloon on its distal end. The catheter is inserted in a vein downstream from the occlusion and advanced to the occluded region, at which time the balloon is inflated to stop the normal flow of blood and the medical agent injected. When sufficient time has elapsed for the medical agent to have its desired effect, the balloon is deflated and normal blood flow restored. This, of course, means that any remaining medical agent will be washed from the treatment area and carried to the lungs, kidneys and liver where it is discharged or metabolized.

In another approach to localized delivery of medical agents, Freeman, et al., J. App. Phys., Supp. Vol. 31, 404S-405S (May 1960), proposed using medical agents combined with iron particles. A powerful magnet is positioned at or near the target site to immobilize the iron laden medical agent. Later, Myers, et al., Amer. J. Roentg., 90, 1068-1077 (November 1963), suggested iron carbonyl, rather than the metal, particles as the carriers of the therapeutic agents. In either case, when the magnetic field is removed, the particles are free to move with the flow of blood away from the target site. Any residual medical agent still bound to the particles would become essentially systemic.

In U.S. Pat. No. 4,345,588, Widder, et al., describe magnetic biodegradable microspheres containing a medical agent. The microspheres are intravascularly administered and magnetically localized in a target capillary bed at which time the medical agent is released. Widder's microspheres are 0.5 to 1.5 microns in size. Anything bigger could occlude the capillary system and cause ischemia of the surrounding tissue. Once a sufficient period of time has elapsed for the medical agent to accomplish its purpose, the magnetic field is removed and the microspheres, and any residual therapeutic agent contained therein, are released into the circulatory system.

Many other approaches to delivering medical agents to specific areas of the body have been described. However, none of them attempts to deal with what happens to residual medical agent once the localizing influence is removed and the medical agent leaves the target area and becomes systemic. As was mentioned at the outset, there are many therapeutic and diagnostic agents that are beneficial at a selected target in a patient's body but are harmful if distributed systemically. For example, without limitation, gene therapy, chemotherapy and radiation therapy agents are effective locally but potentially dangerous if allowed to become systemic. Even fairly common medical agents such as antibiotics, when administered at the concentration required to treat certain disorders, can be detrimental to the well-being of a patient if allowed access to non-target areas or organs. For example, diabetes, a disease that causes restricted blood flow and arterio-venous shunting in limbs, can cause diabetic ulcers, usually on a patient's extremities, e.g., the feet. Treatment of diabetic ulcers usually involves systemic administration of massive amounts of antibiotics, either orally or by venous injection. Unfortunately, the concentration of antibiotic needed to treat the infection often results in toxemia in other areas or organs of the body. A few approaches for preventing undesirable systemization of medical agents have been described.

For example, in U.S. Pat. No. 4,192,302, Boddie discloses a device for isolating blood flow through the liver such that the blood can be loaded with extremely high doses of a chemotherapeutic agent and circulated through the liver only. The liver can, in this manner, be perfused with the chemotherapeutic agent while avoiding any toxic effects in other parts of the body. Bodden, in U.S. Pat. No. 5,069,662, describes a similar but somewhat simpler device for accomplishing the same thing.

In addition to therapeutically and diagnostically useful medical agents that are intentionally administered to patients through the vascular system, many undesirable agents resulting from, for example without limitation, injury or disease, often find their way into the vascular system and, through the vascular system, become systemic. Examples of such undesirable agents include, without limitation, low-density lipoproteins (LDL, cholesterol), allergy-causing antigens and antibodies, pathogens such as bacteria, viruses and prions, metastasizing cancer cells and heavy metals such as mercury and lead. Various approaches to removing such undesirable agents from the circulation have been described.

For instance, in U.S. Pat. No. 3,959,128, Harris describes a process for removing endotoxins from blood comprising passing contaminated blood through an extra-corporeal column containing a non-ionogenic, hydrophobic, non-polar aliphatic resin capable of adsorbing the endotoxins.

In U.S. Pat. No. 4,820,261, Schmoll, et al., describe a similar system for removing tumor-generated substances from a patient's blood by withdrawing blood from a vein that drains the region of the body containing the tumor and pumping it through a column containing immobilized antibodies.

In U.S. Pat. No. 4,816,409, Tanaka, et al., disclose a device for removing tumor cells from blood by contacting the blood with water-insoluble anti-tumor monoclonal antibodies in an extra-corporeal container.

In U.S. Pat. No. 4,464,165, Pollard describes a method for removing IgG immunoglobulins and immune complexes from whole blood. The blood is contacted with an inactivated protein A-bearing Staphlococcus aureus bacteria immobilized on an extra-corporeal polymeric matrix.

Devices and methods, such as those described above, are generally less than optimal due to the potential for blood damage caused by the extra-corporeal tubing and pump, the dilution of the patient's blood by the circuit priming solution and the need for large quantities of anticoagulant to prevent clotting. Careful attendance of the machine by a perfusionist is also required to prevent accidents.

In addition to the above affinity separation techniques, a number of intra- and extra-corporeal mechanical filtering devices and methods have been described.

For example, mechanical filters have been developed for removing large emboli, such as blood clots that break loose from thromboses of deep leg veins, that threaten the lungs and heart. These filters are usually placed in the vena cava. In practice, such intravascular mechanical filters are generally limited to removing emboli greater than about 1 millimeter in diameter.

In U.S. Pat. No. 4,873,978, Ginsburg describes a vascular catheter that has a wire mesh strainer at its distal end. The catheter is positioned downstream from the site of an angioplasty to capture embolic particles released by the treatment.

Emboli filters, such as those describe above, are extensively used in extravascular devices such as heart-lung machines. However, mechanical filters placed in the blood stream have physical limits to the size of the emboli that they can remove because of the presence of cellular blood components. Blood cells range in size from 2 to 12 microns. Attempting to use filters with an effective pore size less than about 25 microns can result in plugging of the filter element with blood cells. Although heparin coating the filter will reduce the tendency of platelets to aggregate on the filter, the limitation is still a problem.

In U.S. Pat. No. 4,261,828, Brunner, et al., disclose an apparatus for detoxifying blood comprising a container in which large, generally spherical particles that are blood compatible but non-permeable to corpuscular blood components are placed. Smaller particles containing active ingredients capable of capturing or neutralizing certain soluble toxic substances in the blood are embedded in the large particles. As the serum portion of the blood flows through the container, it permeates the large particles and comes in contact with the smaller particles containing the active agent that removes the toxic substances.

From the above, it is clear that there remains a need for devices and methods that are capable of capturing or neutralizing medical agents within a patient's vascular system. The present invention provides such devices and methods. In addition, the devices and methods disclosed herein may be used to activate medical agents at target locations in a patient's body.

### SUMMARY OF THE INVENTION

Thus, one aspect of the present invention is a catheter device for modulating a medical agent in a patient's body comprising a first elongate shaft having a proximal and a distal end and a medical agent modulating means operatively coupled to the distal end of the shaft.

An aspect of this invention is a catheter devise further comprising a lumen disposed within the first shaft, the lumen extending axially from the proximal end of the shaft to its distal end and a second elongate shaft having a proximal end and a distal end, the second shaft being extendably and retractably disposed within the lumen of the first shaft.

An aspect of this invention is a catheter device further comprising a flow restriction means operatively coupled to the distal end of the first shaft.

An aspect of this invention is a catheter device wherein the flow restricting means comprises an inflatable balloon.

An aspect of this invention is a catheter device wherein the medical agent modulating means comprises a capture means.

An aspect of this invention is a catheter device wherein the medical agent comprises a magnetically sensitive carrier and the capture means comprises one or more magnets.

An aspect of this invention is a catheter device wherein the medical agent comprises a tumor cell antibody and a chemotherapeutic agent.

An aspect of this invention is a catheter device wherein the medical agent comprises a tumor cell antibody and a radiation therapy agent.

An aspect of this invention is a catheter device comprising magnetic flux focusing means operatively coupled to the magnet or magnets.

The magnetic flux focusing means comprises ribbed cores in an aspect of this invention.

The magnet(s) comprise electromagnets in an aspect of this invention.

The capture means comprises an antibody in an aspect of this invention.

The capture means comprises an antigen in an aspect of this invention.

The capture means comprises biotin in an aspect of this invention.

The capture means comprises avidin in an aspect of this invention.

The capture means comprises a ligand in an aspect of this invention.

The capture means comprises a ligand receptor in an aspect of this invention.

The medical agent modulating means comprises a neutralizing means in an aspect of this invention.

The neutralizing means comprises an enzyme in an aspect of this invention.

The neutralizing means comprises a hormone in an aspect of this invention.

The medical agent modulating means comprises an activating means in an aspect of this invention.

The activating means comprises an enzyme in an aspect of this invention.

The activating means comprises a cytokine in an aspect of this invention.

The cytokine comprises interleukin-2 in an aspect of this invention.

An aspect of this invention is a catheter device further comprising a medical agent delivery means operatively coupled to the distal end of the second shaft.

The delivery means comprises a cannula in an aspect of this invention.

An aspect of this invention is a catheter device wherein the medical agent modulating means is detachable from the distal end of the first shaft.

An aspect of this invention is a catheter device wherein the medical agent modulating means is re-attachable to the distal end of the first shaft.

The detachable means comprises a fiber bundle in an aspect of this invention.

The fiber bundle comprises a ferromagnetic or superparamagnetic material in an aspect of this invention.

An aspect of this invention is a method for modulating a medical agent in a patient's body comprising inserting a catheter device into a vein that drains blood from a target in a patient's body, the catheter device comprising a first flexible elongate shaft, the shaft comprising a proximal end and a distal end and a medical agent modulating means operatively coupled to the distal end of the first shaft; injecting the medical agent into an artery that supplies blood to the target; and, modulating the medical agent with the medical agent modulating means.

An aspect of this invention is a method for modulating a medical agent in a patient's body comprising inserting a catheter device into a vein that drains blood from a target in a patient's body, the catheter device comprising a first flexible elongate shaft having a proximal end, a distal end and a lumen disposed axially in the shaft from the proximal end to the distal end, a second flexible elongate shaft having a proximal end and a distal end, the second shaft being extendably and retractably disposed within the lumen of the first shaft, a medical agent modulating means operatively coupled to the distal end of the first shaft and a medical agent delivery means operatively coupled to the distal end of the second shaft; extending the second shaft to the target; delivering the medical agent to the target; and, modulating the medical agent with the medical agent modulating means.

An aspect of this invention is a method for modulating a medical agent in a patient's body comprising inserting a catheter device into a body cavity of a patient, the catheter device comprising a first elongate shaft having a proximal end, a distal end and a lumen disposed axially within the shaft from the proximal end to the distal end, a second shaft having a proximal end and a distal end, the second shaft being extendably and retractably disposed within the lumen of the first shaft, a medical agent modulating means operatively coupled to the distal end of the first shaft and a medical agent delivery means operatively coupled to the distal end of the second shaft; extending the second into the body cavity; delivering the medical agent into the body cavity and modulating the medical agent with the medical agent modulating means.

An aspect of this invention is the above methods in which the catheter device comprises a flow restriction means operatively coupled to the distal end of the first shaft.

An aspect of this invention is the above methods in which the flow restricting means comprises an inflatable balloon.

An aspect of this invention is the above methods further comprising detaching the modulating means from the first shaft at the target, modulating the medical agent and then re-attaching the modulating means to the distal end of the first shaft.

An aspect of this invention is the above methods in which the modulating means comprises a fiber bundle.

An aspect of this invention is the above methods in which the fiber bundle comprises a ferromagnetic or superparamagnetic material.

An aspect of this invention is the above methods in which the medical agent modulating means comprises a capture means.

An aspect of this invention is the above methods in which the medical agent comprise a magnetically sensitive carrier and the capture means comprises one or more magnets.

An aspect of this invention is the above methods in which the medical agent further comprises a tumor cell antibody and a chemotherapeutic agent.

An aspect of this invention is the above methods in which the medical agent further comprises a tumor cell antibody and a radiation therapy agent.

An aspect of this invention is the above methods further comprising a magnetic flux focusing means operatively coupled to the one or more magnets.

An aspect of this invention is the above methods in which the magnetic flux focussing means comprises ribbed cores.

An aspect of this invention is the above methods in which the magnet(s) are electromagnets.

An aspect of this invention is the above methods in which the capture means comprises an antibody.

An aspect of this invention is the above methods in which the capture means comprises an antigen.

An aspect of this invention is the above methods in which the capture means comprises biotin.

An aspect of this invention is the above methods in which the capture means comprises avidin.

An aspect of this invention is the above methods in which the capture means comprises a ligand.

An aspect of this invention is the above methods in which the capture means comprises a ligand receptor.

An aspect of this invention is the above methods in which the medical agent modulating means comprises a neutralizing means.

An aspect of this invention is the above methods in which the neutralizing means comprises an enzyme.

An aspect of this invention is the above methods in which the neutralizing means comprises a hormone.

An aspect of this invention is the above methods in which the modulating means comprises an activating means.

An aspect of this invention is the above methods in which the activating means comprises an enzyme.

An aspect of this invention is the above methods in which the activating means comprises a cytokine.

An aspect of this invention is the above methods in which the cytokine comprises interleukin-2.

### DETAILED DESCRIPTION OF THE INVENTION

### BRIEF DESCRIPTION OF THE DRAWINGS

FIG. 1 is a schematic representation of a treatment area (the target) showing a medical agent delivery catheter located in an artery serving the target and an extended surface area medical agent capture means located within a vein that drains the target area.

FIG. 2 is a schematic representation of a section of the extended surface area of the medical agent capture means of FIG. 1.

FIG. 3 is a schematic representation of a catheter incorporating an electromagnet to deliver and capture a medical agent. The catheter is shown located within a vein that drains the target area.

FIG. 4 is a schematic representation of a blood vessel in which a catheter containing a collapsed, deployable medical agent capture means has been deployed.

FIG. 5 is a schematic representation a blood vessel in which a catheter deploying a medical agent capture means is depicted.

FIG. 6 is a schematic representation of a blood vessel containing a deployed medical agent capture means.

FIG. 7A is a schematic representation of the distal portion of a catheter incorporating permanent magnets to capture a medical agent, shown in a collapsed position.

FIG. 7B is a schematic representation of the distal portion of the catheter shown in FIG. 7A, shown in an expanded position.

FIG. 8 is a schematic representation of the distal portion of an elongated wire incorporating permanent magnets to capture a medical agent, shown in an expanded position.

FIG. 9 is a schematic representation of the distal portion of another catheter embodiment, incorporating permanent magnets to capture a medical agent, shown in an expanded position.

FIG. 10A is a schematic representation of a catheter incorporating an electromagnet with focusing ribs to deliver and capture a medical agent. The catheter is shown located within a vein that drains the treatment area.

FIG. 10B is a cross sectional view of a focusing rib of FIG. 10A.

FIG. 11 is a schematic representation of a catheter incorporating permanent magnets with focusing ribs to deliver and capture a medical agent. The catheter is shown located within a vein that drains the treatment area.

### Definitions

As used herein, a “catheter device” refers to a thin, elonagate shaft, which may be solid or may contain a lumen. The shaft may take any number of shapes, when viewed in cross-section, such as, without limitation, a square, a rectangle, an oval, an ellipse, a trapezoid or a circle. For example, if the cross section is circular and the shaft contains a lumen, it would simply be a tube. The shaft may be inserted into a body cavity, duct or vessel to provide access to that location for carrying out a variety of medical procedures. If it contains a lumen, the device may include one or more additional elongate shafts within the lumen of the first tube. The various shafts may independently be flexible or rigid.

As used herein, the term “medical agent” refers to any substance that when found in the body affects the health or well-being of the patient. Medical agent includes, without limitation, therapeutic and diagnostic agents.

A “therapeutic agent,” for the purposes of this invention, means a chemical substance that has a beneficial effect on the health and well-being of a patient. Examples, without limitation, of therapeutic agents include antibiotics, cancer chemotherapeutics, gene therapy agents, antibiotics, antineoplastics, hormones, proteins, peptides, lectins, antibodies, antivirals, radiation sources such as cobalt, radium, radioactive iodine, etc., anticoagulants, enzymes, hepatoprotectants, vasodilators, nitric oxide and the like.

A “diagnostic agent,” for the purposes of this invention, refers to a chemical substance that is used to facilitate the diagnosis of a condition, disorder or disease in a patient. Examples, without limitation, of diagnostic agents are imaging agents such as a radionuclides (e.g. In-111, Tc-99m, I-123, I-125 F-18, Ga-67, Ga-68), atoms having electrons with unpaired spins (e.g. Fe, Gd and the lanthanides), free radicals and chelated DPTA(manganese).

A medical agent, as used herein, also refers to an undesirable chemical substance that has a detrimental effect on a patient's health and well-being. Thus, such substances as, without limitation, heavy metals, poisons, cancer cells, pathogens such as bacteria, fungal cells, viruses and prions and the chemicals they produce, LDLs and certain other proteins and enzymes are medical agents for the purposes of this invention.

As used herein, the term “modulating,” refers to affecting a change in a chemical, physical or biological characteristic of a medical agent compared to that characteristic prior to encountering a device and method of this invention. For example, if, prior to encountering a device and method herein, a particular medical agent moves freely through the body, in the vascular system or otherwise, retarding or stopping that movement constitutes modulation. Similarly, if a medical agent were normally immobilized on some surface in the body, releasing it so it moves freely constitutes modulation of a physical characteristic of that agent. If a medical agent is biologically inactive in the body, then rendering it biologically active would constitute modulation. Conversely, if a medical agent were biologically active as found in the body prior to interception with a device and method of this invention, then rendering it inactive would constitute modulation. Based on this disclosure, numerous other modes of modulation will become apparent to those skilled in the art; such modulations are within the scope of this invention.

As used herein, “extendable and retractable” refer to the movement of one tubular shaft within the lumen of another tubular shaft such that the distal end of the second shaft can extended beyond the distal end of the first shaft and can be retracted so that it is proximal to the distal end of the first shaft.

As used herein, a “flow restriction means” refers to any manner of device that is capable of retarding or stopping the normal flow of a fluid in the body. For example, without limitation, any manner of device that retards or stops the flow of blood from the heart through the arteries to the organs, to the veins, to the lungs and finally back to the heart, would constitute a flow restriction means. An example, without limitation, of a flow restriction means is a balloon that, when inserted in an artery or vein and inflated, blocks the flow of blood completely or partially.

As used herein, “capture means” refers to a device or substance that is capable of binding to and immobilizing, temporarily or permanently, a medical agent. Examples, without limitation, of such devices and substances include magnetic attraction, chemical and biological binding interactions such as that between ligand and recepter, antigen and antibody or enzyme and substrate and biochemical attractions such as that between avidin and biotin or a hormone and its substrate.

As used herein, a “magnetically sensitive carrier” refers to a device or substance that can be immobilized at a target in a patient's body by exposure to an magnetic field. The carrier also reversibly binds a therapeutic or diagnostic agent such that the agent can be released once the carrier as reached and been immobilized at the target.

As used herein, a “tumor cell antibody” refers to a chemical substance that recognizes antigens on the surface of tumor cells and binds to them.

As used herein, a “chemotherapeutic” refers to a chemical substance used to treat cancer.

As used herein, a “radiation therapy agent” refers to a substance that releases radioactive particles and thereby kills living cells.

As used herein, a “magnetic flux focussing means” refers to a device that is capable of concentrating a magnetic field in a selected region of a patient's body. Examples, without limitation, of magnetic focussing means are ferromagnetic or paramagnetic substances that have been shaped such that, when a magnetic field is generated in their vicinity, the field is concentrated and directed to a particular location.

As used herein, a “neutralizing agent” refers to a material that deactivates, disables, counteracts or causes a malfunction in a medical agent such that the medical agent no longer affects the health or well-being of a patient.

As used herein an “activating agent” refers to a substance that interacts, directly or indirectly, with a medical agent and, as a result, modulates a chemical, physical or biological characteristic of the agent that renders it capable of exerting an effect on the health or well-being of a patient.

As used herein, a “target” refers to a specific site in a patient's body where it is desired to localize a medical agent. The target may be, without limitation, a specific organ or portion thereof, a particular blood vessel or portion or a body cavity or portion thereof.

As used herein, a “carrier” refers to a device or material used to transport a therapeutic or diagnostic agent to a target site in a patient's body. Examples, without limitation, of carriers, include organic particles, inorganic particles, liposomes, proteins, lipids, polymers, peptides, lipopolymers, biological cells such as virus, bacteria or prions, antibodies, antigens, hydrogels, polymers, dendrimers and the like.

### Discussion

The present invention is described with reference to the attached drawings. However, the invention is not intended, nor is it to be construed, as being in any manner limited to the embodiments in the drawings. That is, variations in the described devices and methods as well as other applications for the devices and methods will become apparent to those skilled in the art based on the disclosures herein. All such variations and applications are within the scope of this invention.

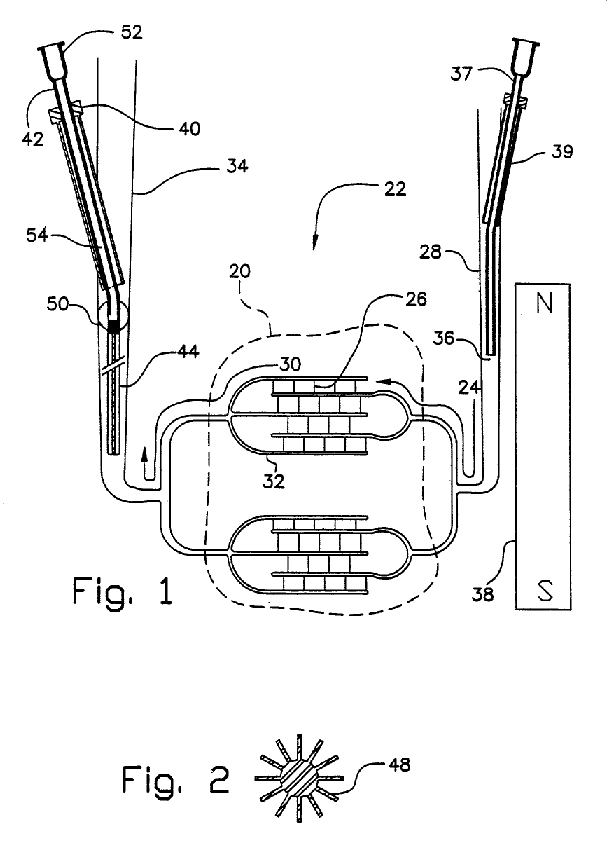

FIG. 1 shows a selected target location 20 within a simplified circulatory system 22. The direction of normal blood flow (arrow 24) is from artery 28 to microcirculatory capillaries 26, to venules (veinlets that are continuous with the capillary system and join to form veins) 32, to vein 34 and eventually to the heart (not shown). A medical agent is attached to magnetically sensitive particles (not shown) that are smaller than 5 microns and the particles are suspended in a physiologically compatible fluid such as normal saline. The suspension is delivered into artery 28 at injection site 36 through delivery catheter 37 that has been placed in the artery through introducer sheath 39. For best results, the magnetically sensitive particles are biocompatible, nontoxic and non-immunogenic. Many such particles are known in the art and any of these could be used with the devices and methods of this invention. It may also be advantageous to have particles that are detectable by non-invasive means such as fluoroscopy, magnetic resonance imaging (MRI) or ultrasound. Again, such detectable particles are known in the art and may be used with the present invention.

If the location of artery 28 is such that direct introduction of catheter 37 is not convenient, catheter 37 may be introduced into a remote artery and navigated to injection site 36.

To localize the magnetically sensitive particles at target 20, magnet 38 is positioned such that a magnetic field is created in the vicinity of capillaries 26. The magnetically sensitive particles can thus be immobilized in the capillaries for a predetermined period of time during which the medical agent can be released to have its desired effect. Once an appropriate time period has passed, magnet 38 is turned off, if it is an electromagnet, or removed from the area if it is a permanent magnet, which releases the particles which then proceed to move with the flow of blood. However, prior to disengaging the magnetic field, sheath 40 is placed in vein 34 in a retrograde direction and recovery catheter 42 is inserted into the sheath. Catheter 42 includes distal segment 44, which contains a medical agent capture means of this invention. When the medical agent carriers are magnetically sensitive particles, distal segment 44 would comprise one or more permanent or electromagnets, or a combination thereof. For example, without limitation, distal segment 44 could be a device having a cross-section such as that shown in FIG. 2. Radial fins 48 provide an extensive surface area on which to capture the magnetically sensitive particles. Fins 48 could be impregnated with magnetic particles or lengths of ferromagnetic material wound with wire to create electromagnets. Expandable balloon 50 is affixed to catheter 42 proximal to distal segment 44. Luer fitting 52 communicates with balloon 50 by means of lumen 54 in catheter 42. At the end of the treatment time and just before the localizing magnetic field is released, the balloon is inflated with fluid through a syringe attached to luer fitting 52 sufficiently to retard the passage of blood pass the balloon. Magnetically sensitive particle laden blood passes slowly by distal segment 44 containing magnet or magnets. The magnetically sensitive particles are captured and immobilized by the magnet(s) and will be removed from the patient's circulatory system when the catheter is withdrawn.

In another embodiment of this invention, the blood flow restricting means comprises an external pressure cuff. This would be particularly appropriate when target area 20 is located in an extremity; i.e., an arm or a leg. For example, without limitation, such device could be employed to retain bone morphogenic protein (BMP) at the site of a leg fracture (the target). Referring again to FIG. 1, catheter 42 would be inserted into femoral vein 34. Magnetically sensitive particles carrying BMP would be injected into femoral artery 28 of the broken leg and would be carried by the blood to the capillaries in the vicinity of the fracture. There the particles would be immobilized by an external magnet 38 located in proximity to the fracture and BMP could be released. After an appropriate treatment period, a pressure cuff would be placed on the leg above the fracture site and inflated to stop the flow of blood from the leg. The magnetic field is then removed. The pressure cuff is released enough that blood is permitted to slowly flow past catheter distal segment 44, which, as above, consists of a magnet or group of magnets. The magnetically sensitive particles are attracted to and immobilized by the magnet(s). Catheter 42 and the immobilized particles can then be removed from the patient.

In another embodiment of this invention, the magnetic particles comprise magnetically sensitive liposomes and the medical agent is an angiogenic growth factor such as VEGF. Referring again to FIG. 1, target 20 could be, for example, ischemic myocardial heart tissue within the left ventricle of a patient's heart. Artery 28 would then be the left anterior descending artery and vein 34 would be the great cardiac vein. A magnetic field is created within the heart using procedures and devices well known in the art. VEGF-containing liposomes would be delivered to the target, immobilized there by the magnetic field and the VEGF released to promote angiogenesis. After an appropriate period of time, the magnetic field is removed and the liposomes are carried with the flow of blood through draining venules 32 and into cardiac vein 34 where they are collected by the magnet(s) at distal segment 44. By capturing the liposomes in this manner, residual VEGF they might contain would be removed from the general circulation, thereby negating any deleterious effects that the angiogenic growth factor might have in non-target areas of the body. For example, the factor would not be available to promote growth of a nascent or undetected tumor. Magnetic liposomes useful in this embodiment of the present invention are discussed by Kubo T., et al., “Targeted delivery of anticancer drugs with intravenously administered magnetic liposomes in osteosarcoma-bearing hamsters,” Int J. Oncol. , 2000, 17:309-315.

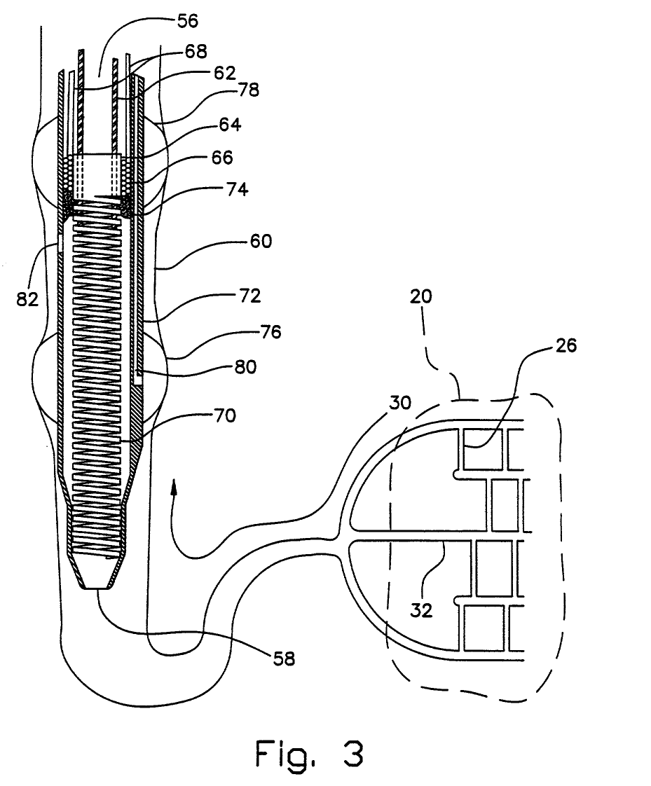

Another non-limiting embodiment of the present invention is shown in FIG. 3 where retrograde venous delivery of a medical agent is depicted. Retrograde administration permits the use of larger carrier particles, up to about 20 microns. Such particles would still be able to penetrate venules due to their thin, highly porous walls. Retrograde administration has several advantages over standard intravenous administration. For one, it avoids circulation through the liver. The liver is a natural filter, designed to remove particles such as the ones used herein. Some of the particles and the drug they are carrying could thus be removed by the liver and never reach the target. In addition, the drug-carrying particles would be concentrated in the liver where the drug might have a deleterious effect. Furthermore, retrograde administration means that only one vascular cannulation is required for delivery and capture of the particles. Finally, the larger particles render magnetic immobilization in the target tissue and eventual capture easier and more efficient.

In FIG. 3, tube 62, which extends most of the length of the catheter, terminates slightly inside tube 64. Tube 64 is constructed of a ferromagnetic material and is the core of an electromagnet. Wires 66 are wrapped around tube 64. Wire ends 68 are connected to a power source (not shown), which, when activated, creates a current through wires 66 and, as a result, a magnetic field around tube 64. Spiral coil 70 is cut into the distal end of tube 64 and provides a large surface area as well as a tortuous path to blood flow. This combination increases the likelihood that the magnetically sensitive carriers are captured in tube 64. Many configurations of the magnet other than a spiral coil could be used to accomplish capture of the particles. For example, without limitation, ferromagnetic fiber bundles such as steel wool, a screen mesh formed of woven or braided ferromagnetic fibers or round or irregularly shaped ferromagnetic beads, etc., could be used. Any such configuration is within the scope of this invention.

Tube 62, and the electromagnet components are incorporated within outer catheter shaft 72 and are bonded together as a unit by medical adhesive 74 which also serves to isolate blood passing through spiral coil 70 from the electromagnet wire winding 66. Inflatable balloons 76 and 78 are affixed to the outside of catheter shaft 72. Balloon 78 is in communication with a syringe (not shown) via lumen 80. Likewise, balloon 76 is in communication with its own syringe (also not shown) through a second lumen (not shown) within catheter shaft 72. Thus, inflation and deflation of the two balloons may be independently controlled. Catheter shaft 72 contains one or more blood flow exit holes 82 (only one can be seen in FIG. 3).

A non-limiting exemplary illustration of the use of a device such as that shown in FIG. 3 for retrograde delivery and then capture of a drug would be the administration of a chemotherapeutic agent using magnetically sensitive liposomes. A typical procedure might be the following:

After placement of distal end 58 of saline filled catheter 72 in vein 60, balloon 78 is inflated until a seal is created against the inner wall of vein 60. A biocompatible solution of magnetically sensitive liposome carriers containing adriamycin is injected through lumen 56, exits opening 58 and flows retrograde into venules 32. If desired, a magnet (not shown) can be place adjacent to target area 20 to further localize the liposomes. Additionally, by proper choice of liposome transition temperature, the adriamycin could be controllably released by localized heating induced by an oscillating electromagnetic field.

At the end of the treatment period, the electromagnet comprising spiral coil 70 and wire winding 66 is energized. If a magnet has been placed next to target area 20, it is removed. Balloon 76 is inflated, sealing against the inner wall of vein 60 and then balloon 78 is slowly deflated, allowing blood and liposome carriers to flow antegrade (i.e., in the normal direction of blood flow, indicated by arrow 30). Blood enters catheter 72 through opening 58, flows around spiral coil 70 and exits through holes 82. The magnetically sensitive liposome carriers are captured and immobilized on electromagnet spiral coil 70. Balloon 76 is then deflated and the catheter and captured liposome carriers withdrawn from the patient.

Alternately, balloon 78 could be inflated and the electric current in winding 66 stopped. The liposomes are released and proceed through lumen 56. A vacuum is applied at the proximal end of lumen 56 and blood containing the liposomes is withdrawn through the lumen. In this manner, the procedure can be repeated through as many additional cycles of liposome particle delivery and capture as desired without need for removal of the catheter.

Depending on the location of the vein 60, other methods are available to produce an on-demand magnetic field within the delivery and recovery catheter. If the catheter is located near the surface of the patient body, the electromagnet could be replaced by ferromagnetic material such as 430 stainless steel wire. If desired, the wire could be woven into a mesh that would be positioned perpendicular to the blood flow for particle capture. Creating a magnetic field outside the patient's body but near the location of the ferromagnetic material would produce a corresponding magnetic field in the mesh, which would capture the particles.

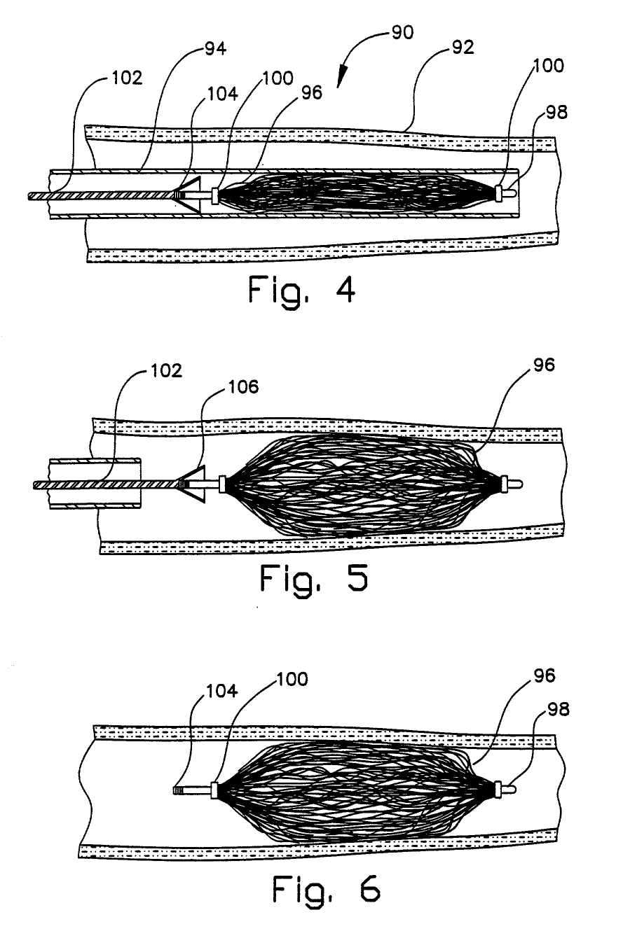

FIGS. 4-6 show how an embodiment of this invention could be employed in IL-2 therapy.

Interleukin-2 (IL-2) is produced by lymphocytes. It activates lymphokine-activate killer (LAK) cells which kill tumor cells and improve the recovery of some immune functions in certain immuno-deficiency disorders. Administration if IL-2 has also been shown to be effective in treating some forms cancers such as melanoma and non-Hodgkin's lymphoma. Unfortunately IL-2 therapy can cause serious side effects including liver dysfunction, central nervous system disorders and kidney failure. Thus a means for localizing its effects and removing it before it can become systemic and reach the liver or kidneys would be extremely desirable.

FIG. 4 shows a distal segment of a single lumen catheter 94 containing a compressed bundle of fibers 96 that are attached at each end of the bundle to rod 98 by band 100. Rod 98 is attached to delivery shaft 102 by a threaded portion 104.

The catheter is placed within an artery or vein of a patient using a standard Seldinger technique, that is, by first inserting an introducer sheath(not shown) followed by catheter 94. The catheter 94 may be placed in an antigrade or retrograde orientation.

Bundle 96 is deployed by retracting catheter 94 as shown in FIG. 5. Bundle 96 is composed of a biocompatible polymer or metal fibers that are bent or heat set in a random configuration. Upon retraction of catheter 94, bundle 95 expands and fills the blood vessel. The fibers of the bundle are coated or impregnated with IL-2 that is released over a period of time in the vessel.

FIG. 6 shows bundle 96 released from rod 98. This is accomplished by spinning delivery shaft 102 to unscrew it from rod 98 at thread 104, after which the catheter and delivery shaft can be withdrawn leaving bundle 96 in place. For long term placement it is desirable to also coat a portion of the fibers with an antithrombogenic material such as Carmeda® BioActive Surface immobilized heparin or similar material. The bundle may be recovered by reversing the procedure with cone 106 aiding in aligning the threads for re-attachment. Cone 106 and rod 98 can be made of a radiopaque material known in the art in order to visualize them by fluoroscopy.

Another example of an embodiment of this invention would involve the strong affinity of biotin for avidin, which could be exploited to assist in the use of radiolabeled monoclonal antibodies (Mabs) for the diagnosis and treatment of malignant tumors. That is, the Mabs could be biotinylated prior to administration. After circulation and uptake of the Mabs by the tumor, any remaining Mabs could be removed from the circulation by capturing them with an avidin coated bundle such as that shown in FIGS. 4-6.

The bundle approach could also be employed to capture and immobilize magnetically sensitive particles. In this embodiment, the bundle could comprise superparamagnetic particles. When such particles are exposed to an external magnetic field, they are magnetized but when the external field is removed, they revert completely to an unmagnetized state, that is, they retain none of the magnetism when the external field is removed. Thus, the bundle could be energized by an external permanent or electromagnet to produce a strong magnetic field within the blood vessel and thereby trap and immobilize magnetically sensitive medical agent carrier particles.

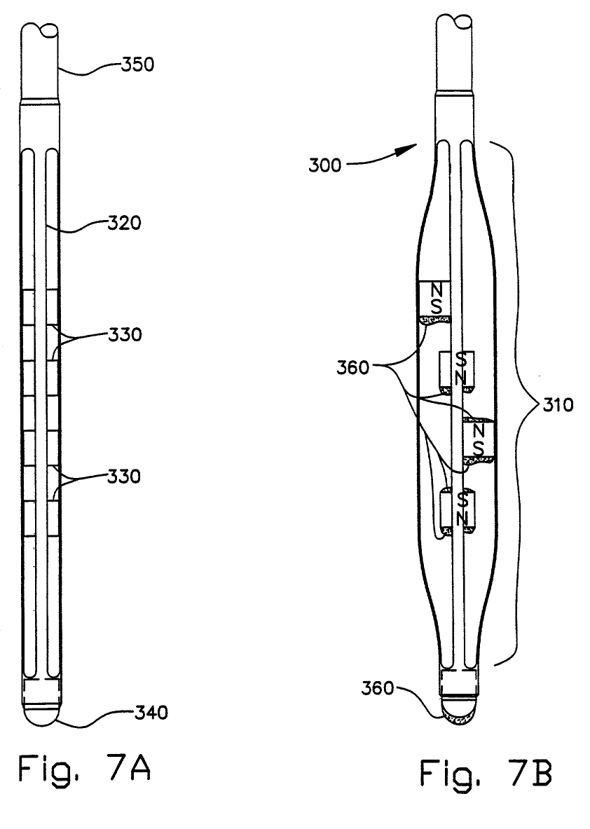

FIGS. 7A and 7B depict another embodiment of this invention. This embodiment relates to a segment 310 of a tube which consists of four ribs 320 (one rib is directly behind the rib in the center of the figure and is not shown), to each of which is bonded one or more magnet(s) 330. In FIG. 7A, ribs 320 are shown in a compressed state as they would exist when the segment is contained within a standard catheter guide or access sheath. As such, the segment can be advanced into any size blood vessel that the guide can traverse. Once the device is in place, the guide or sheath is withdrawn and ribs 320 automatically expand and contact the vessel wall at which time magnets 360 are deployed as shown in FIG. 7B within the blood vessel. The magnets are positioned such that they present a staggered appearance when viewed axially along the length of the segment. A fifth magnet 340 can be attached to the distal end of segment 310. Magnetically sensitive particles 360 are captured and immobilized by magnets 330 and 340. Preferably, the poles of the magnets are oriented parallel to the long axis of the segment. In this configuration, the captured magnetically sensitive particles, which will concentrate at the poles as shown in FIG. 7B, will not be scraped off magnets 330 when segment 310 is withdrawn back into the guide catheter and recompressed. Ribs 320 may be any suitable biocompatible, elastic material such as 316 stainless steel, super-elastic nickel-titanium alloys, polycarbonate resin, etc. Typically, magnets 330 will have sufficient radio-opacity to be viewed by conventional fluoroscopic equipment to assure proper deployment. They may also be visualized by MRI imaging systems. In order to effectively capture small magnetically sensitive particles, magnets 330 are preferably rare earth type magnets containing such materials as neodymium iron boron or samarium cobalt. A suitable thin coating of a material such as PTFE, Chromium, polyolefin polymer or the like on the magnets can be used to insure biocompatibilty and prevent corrosion.

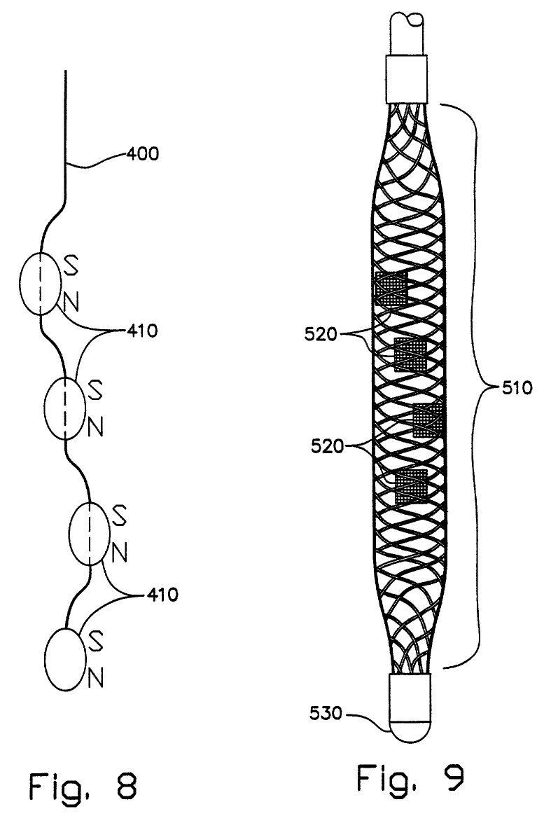

In FIG. 8, magnets 410 are disposed along guide wire 400. Wire 400 is made of a material of sufficient stiffness to permit it to be advanced through a guide catheter along with magnets 410. The wire is bent into a spiral in the region where the magnets are attached. This allows the magnets to collapse to a smaller diameter so that they fit through the guide catheter or introducer sheath. Guide wire 400 would be extended beyond the distal end of a catheter such that magnets 410 are deployed in the blood stream. There they would capture magnetically sensitive particles after which they could be withdrawn back into the catheter along with guide wire 400.

FIG. 9 shows an alternative version of the device shown in FIGS. 7A and 7B. Here distal segment 510 comprises a braided wire mesh instead of discrete struts. Magnets 520 would be attached to the interior portion of the wire mesh. An additional magnet 530 could be positioned at the distal end of the segment just as was the case in the device of FIG. 7A. As were the struts of FIG. 7, the mesh of FIG. 9 would be stored in a guide catheter or access sheath so as to minimize its cross-section and allow advancement into relatively small blood vessels. Once at the desired location, segment 510 would be advanced beyond the distal end of the guide at which time the wire mesh would expand until it contacts the vessel wall. The deployed magnets would then be available to capture magnetically sensitive particles. After the magnetically sensitive medical agent carriers are immobilized on the magnets 520 and 530, the distal segment could be withdrawn back into the catheter or introducer sheath and removed from the vessel.

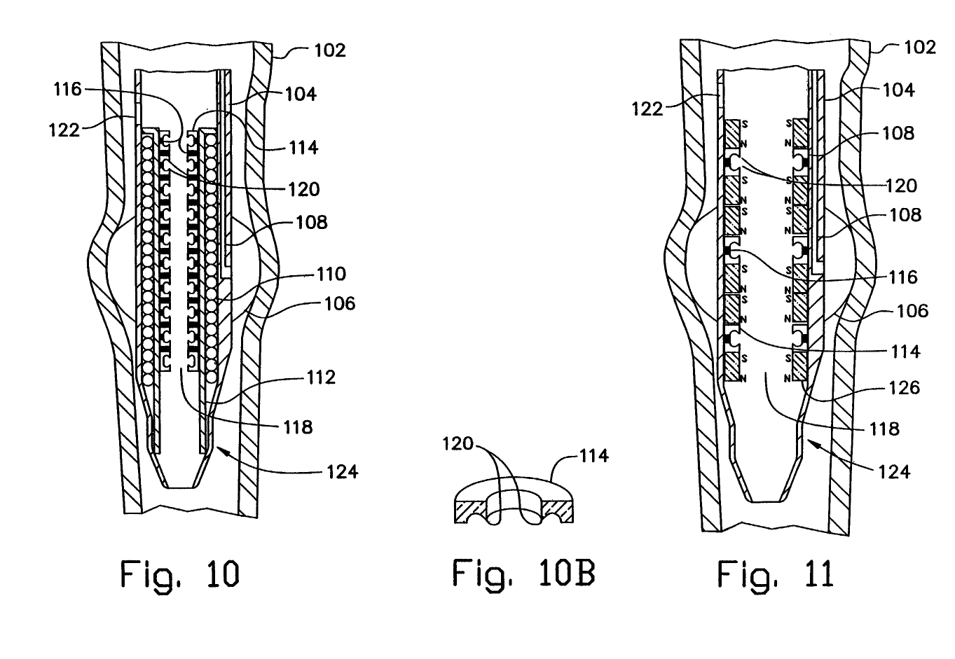

FIG. 10 provides yet another embodiment of this invention. In FIG. 10, the distal end of catheter 124 is shown deployed in vein 102. Blood flow around catheter 124 is prevented by balloon 106, which is inflated through lumen 108 in catheter wall 104. Blood carrying magnetically sensitive particles will flow through central duct 118 and exit through side hole 122 in catheter wall 104. Wall 112 isolates field coil 110 of an electromagnet. As shown, the electromagnet consists of multiple circular ferrite cores 114. Each core 114 is essentially a flattened torus that has been hollowed out on the inside circumference and then cut in half, as shown in FIG. 10B. Each half torus is separated from its other half by an insulator 116. Each core (two halves 114 separated by an insulator 116) is separated from each other core by another insulator 116. The edges 120 of each half torus function as focussing ribs to concentrate the magnetic flux at the edges of the magnets where magnetically sensitive particles will first encounter the magnets. This arrangement of segmented cores 114 and insulators 116 affords excellent particle retention while retaining a flexible catheter tip.

FIG. 11 illustrates a similar design using permanent magnets 126 to replace the electromagnet coil winding of FIG. 10. Each magnet 126 is a flattened torus such as that described above except that the inside circumference of the torus is not hollowed out. Magnets 126 are oriented such that their north and south poles concentrate magnetic flux across focusing ribs 120 located on torus-shaped cores 114. Again, torus-shaped insulators 116 separate cores 114. This embodiment also provides excellent capture of magnetic particles while retaining good catheter flexibility.

### Conclusion

Thus, it will be appreciated that the present invention provides novel devices and methods for the modulation of medical agents in a patient's body through the use of readily manipulated medical agent capture, neutralization or activation means coupled to a catheter.

Other configurations of devices, other capture, neutralization and activation means and other applications of the methods described herein for the modulation of medical agents in a highly localized portion of a patient's body will become apparent based on the disclosures herein. Such other devices, means and applications are within the scope of this invention.

Additional embodiment of this invention are presented in the claims that follow.

What is claimed:

1. A catheter device for modulating a medical agent in a patient's body comprising:
a first elongate shaft having a proximal and a distal end; and,
a medical agent modulating means operatively coupled to the distal end of the shaft.

2. The device of claim 1, further comprising:
a lumen disposed within the first shaft, the lumen extending axially from the proximal end of the shaft to its distal end; and,
a second elongate shaft having a proximal end and a distal end, the second shaft being extendably and retractably disposed within the lumen of the first shaft.

3. The device of claim 1 or 2, further comprising a flow restriction means operatively coupled to the distal end of the first shaft.

4. The device of claim 3, wherein the flow restricting means comprises an inflatable balloon.

5. The device of claim 1, wherein the medical agent modulating means comprises a capture means.

6. The device of claim 5, wherein the medical agent comprise a magnetically sensitive carrier and the capture means comprises one or more magnets.

7. The device of claim 6, wherein the medical agent further comprises a tumor cell antibody and a chemotherapeutic agent.

8. The device of claim 6, wherein the medical agent further comprises a tumor cell antibody and a radiation therapy agent.

9. The device of claim 6, further comprising magnetic flux focusing means operatively coupled to the magnet or magnets.

10. The device of claim 9, wherein the magnetic flux focusing means comprises ribbed cores.

11. The device of claim 6, wherein the magnet(s) comprise electromagnets.

12. The device of claim 5, wherein the capture means comprises an antibody.

13. The device of claim 5, wherein the capture means comprises an antigen.

14. The device of claim 5, wherein the capture means comprises biotin.

15. The device of claim 5, wherein the capture means comprises avidin.

16. The device of claim 5, wherein the capture means comprises a ligand.

17. The device of claim 5, wherein the capture means comprises a ligand receptor.

18. The device of claim 1, wherein the medical agent modulating means comprises a neutralizing means.

19. The device of claim 18, wherein the neutralizing means comprises an enzyme.

20. The device of claim 18, wherein the neutralizing means comprises a hormone.

21. The device of claim 1, wherein the medical agent modulating means comprises an activating means.

22. The device of claim 21, wherein the activating means comprises an enzyme.

23. The device of claim 21, wherein the activating means comprises a cytokine.

24. The device of claim 23, wherein the cytokine comprises interleukin-2.

25. The device of claim 2, further comprising a medical agent delivery means operatively coupled to the distal end of the second shaft.

26. The device of claim 25, wherein the delivery means comprises a cannula.

27. The device of claim 1, wherein the medical agent modulating means is detachable from the distal end of the first shaft.

28. The device of claim 27, wherein the medical agent modulating means is re-attachable to the distal end of the first shaft.

29. The device of claim 27, wherein the detachable means comprises a fiber bundle.

30. The device of claim 29, wherein the fiber bundle comprises a ferromagnetic or superparamagnetic material.

31. A method for modulating a medical agent in a patient's body, comprising:
inserting a catheter device into a vein that drains blood from a target in a patient's body, the catheter device comprising:
a first flexible elongate shaft, the shaft comprising a proximal end and a distal end; and,
a medical agent modulating means operatively coupled to the distal end of the first flexible shaft;
injecting the medical agent into an artery that supplies blood to the target; and,
modulating the medical agent with the medical agent modulating means.

32. A method for modulating a medical agent in a patient's body, comprising:
inserting a catheter device into a vein that drains blood from a target in a patient's body, the catheter device comprising:
a first flexible elongate shaft having a proximal end, a distal end and a lumen disposed axially in the shaft from the proximal end to the distal end;
a second flexible elongate shaft having a proximal end and a distal end, the second shaft being extendably and retractably disposed within the lumen of the first shaft;
a medical agent modulating means operatively coupled to the distal end of the first shaft; and,
a medical agent delivery means operatively coupled to the distal end of the second shaft;
extending the second shaft to the target;
delivering the medical agent to the target; and,
modulating the medical agent with the medical agent modulating means.

33. A method for modulating a medical agent in a patient's body, comprising:
inserting a catheter device into a body cavity of a patient, the catheter device comprising:
a first elongate shaft having a proximal end, a distal end and a lumen disposed axially within the shaft from the proximal end to the distal end;
a second elongate shaft having a proximal end and a distal end, the second shaft being extendably and retractably disposed within the lumen of the first shaft;
a medical agent modulating means operatively coupled to the distal end of the first shaft; and,
a medical agent delivery means operatively coupled to the distal end of the second shaft;
extending the second into the body cavity;
delivering the medical agent into the body cavity; and,
modulating the medical agent with the medical agent modulating means.

34. The method of claim 32, wherein the catheter device further comprises a flow restriction means operatively coupled to the distal end of the first shaft.

35. The method of claim 34, wherein the flow restricting means comprises an inflatable balloon.

36. The method of any of claims 31, 32 or 33, further comprising:
detaching the modulating means from the first flexible shaft at the target;
modulating the medical agent; and,
re-attaching the modulating means to the distal end of the first flexible shaft.

37. The device of claim 36, wherein the modulating means comprises a fiber bundle.

38. The device of claim 37, wherein the fiber bundle comprises a ferromagnetic or superparamagnetic material.

39. The method of any of claims 31, 32 or 33, wherein the medical agent modulating means comprises a capture means.

40. The method of claim 39, wherein the medical agent comprise a magnetically sensitive carrier and the capture means comprises one or more magnets.

41. The method of claim 40, wherein the medical agent further comprises a tumor cell antibody and a chemotherapeutic agent.

42. The method of claim 40, wherein the medical agent further comprises a tumor cell antibody and a radiation therapy agent.

43. The method of claim 40, further comprising a magnetic flux focusing means operatively coupled to the one or more magnets.

44. The method of claim 43, wherein the magnetic flux focussing means comprises ribbed cores.

45. The method of claim 40, wherein the magnet or magnets are electromagnets.

46. The method of claim 39, wherein the capture means comprises an antibody.

47. The method of claim 39, wherein the capture means comprises an antigen.

48. The method of claim 39, wherein the capture means comprises biotin.

49. The method of claim 39, wherein the capture means comprises avidin.

50. The method of claim 39, wherein the capture means comprises a ligand.

51. The method of claim 39, wherein the capture means comprises a ligand receptor.

52. The method of any one of claims 31, 32, or 33, wherein the medical agent modulating means comprises a neutralizing means.

53. The method of claim 52, wherein the neutralizing means comprises an enzyme.

54. The method of claim 52, wherein the neutralizing means comprises a hormone.

55. The method of claim 32, wherein the modulating means comprises an activating means.

56. The method of claim 55, wherein the activating means comprises an enzyme.

57. The method of claim 55, wherein the activating means comprises a cytokine.

58. The method of claim 57, wherein the cytokine comprises interleukin-2.
